(19) United States
(12) Patent Application Publication
(10) Pub. No.: US 2021/0228824 A1
(43) Pub. Date: Jul. 29, 2021

(54) WICKING CAP AND METHODS

(71) Applicant: Aerami Therapeutics, Inc., Durham, NC (US)

(72) Inventors: Ryan Patton, Lafayette, CA (US); Benjamin Stedman, San Jose, CA (US); Blaine Bueche, San Mateo, CA (US); John Patton, San Francisco, CA (US)

(21) Appl. No.: 16/776,304

(22) Filed: Jan. 29, 2020

(51) Int. Cl. A61M 15/00

(52) U.S. Cl. CPC A61M 15/001; A61M 16/142; A61M 15/0025

(57) ABSTRACT

A wicking member and methods of use of the same are disclosed herein. The wicking member can be a component of a medication inhaler. The medication inhaler can include a reservoir bowl defining a reservoir wherein a volume of liquid can be received. The reservoir bowl can include a top, a bottom, and a wall extending from the top to the bottom of the reservoir bowl. The medication inhaler can further include a wicking member proximate to the wall. The wicking member can extend along the wall at least partially from the top of the reservoir bowl to the bottom of the reservoir bowl. The wicking member can wick liquid to the bottom of the reservoir bowl.

### RELATED APPLICATIONS

The present application is related to: U.S. application Ser. No. 13/004,662, filed on Jan. 11, 2011, and entitled “Preservative-Free Single Dose Inhaler Systems”; U.S. application Ser. No. 14/254,128, filed on Apr. 16, 2014, and entitled “Liquid Dispensing And Methods For Dispensing Liquids”; U.S. Application No. 62/019,791, filed on Jul. 1, 2014, and entitled “Aerosolization System With Flow Restrictor And Feedback Device”; U.S. application Ser. No. 14/732,247, filed on Jun. 5, 2015, and entitled “Self-Puncturing Liquid Drug Cartridges And Associated Dispenser”; U.S. application Ser. No. 14/743,711, filed on Jun. 18, 2015, and entitled “Liquid Nebulization System And Methods”; and U.S. application Ser. No. 14/732,446, filed on Jun. 5, 2015, and entitled “Liquid Drug Cartridge And Associated Dispenser”, the entirety of each of which is hereby incorporated by reference herein.

### BACKGROUND

Water has a high surface tension, and thus, the internal cohesive forces of water molecules are strong. These internal cohesive forces draw water molecules together. Due to these forces, water prefers to draw together and form a droplet, because of these internal forces, water in a container interacts with the surface or walls of the container.

In some instances, medication can be delivered systemically by inhalation via a medication-containing mist. While such administering of medication can be advantageous, for example, such medication administration does not use needles, such administration has its own challenges. For example, it can be relatively easier to accurately control dosing of medication administered via one or several pills, or administered with an injection. In light of these challenges, further improvements to devices and methods for delivering medication containing mist are desired.

### BRIEF SUMMARY

One aspect of the present disclosure relates to a medication inhaler. The medication inhaler includes a reservoir bowl defining a reservoir in which a volume of liquid can be received. The reservoir bowl includes a top, a bottom, and a wall extending from the top to the bottom of the reservoir bowl. The medication inhaler includes a wicking member proximate the wall. The wicking member extends along the wall at least partially from the top of the reservoir bowl to the bottom of the reservoir bowl. The wicking member can wick liquid to the bottom of the reservoir bowl. In some embodiments, this liquid can be pulled from the bottom of the reservoir bowl through an orifice. This orifice can, in some embodiments, allow liquid to escape via passive forces such as gravity, or via one or several active forces such as, for example, forces generated by vibration, pressure, and/or pumping.

In some embodiments, the wicking member connects to the wall of the reservoir bowl proximate to the top of the reservoir bowl. In some embodiments, the medication inhaler includes a cap defining an aperture, the cap extending across the top of the reservoir bowl. In some embodiments, the medication inhaler includes a mouthpiece at a front of the medication inhaler and an opposing back of the medication inhaler.

In some embodiments, the cap can have a shape that aligns the wick along an axis of the mouthpiece lumen. In some embodiments, this shape can be, for example, a circular cap with a keying and/or indexing feature, which keying and/or indexing feature can be located on the edge or along the bottom of the cap. In some embodiments, this shape can be a rounded diamond shape with a major axis extending between a cap front and a cap back and a minor axis extending between a cap first side and a cap second side. In some embodiments, the major axis is greater than the minor axis, and wherein the cap is received in the top of the reservoir bowl. In some embodiments, the reservoir bowl has a front proximate to the mouthpiece and a back proximate to the back of the medication inhaler. In some embodiments, the cap is received in the top of the reservoir bowl such that the major axis of the cap extends from the front of the reservoir bowl to the back of the reservoir bowl.

In some embodiments, the wicking member is coupled to the bottom of the cap. In some embodiments, the wicking member extends along the major axis of the cap from the cap front to the cap back. In some embodiments, the wicking member is a single member. In some embodiments, the wicking member can include a plurality of members. In some embodiments, the wicking member includes a first member extending from a cap front and a second member extending from a cap back. In some embodiments, the first member extends at least partially towards the second member.

In some embodiments, the first member intersects the second member. In some embodiments, the first member is coupled to the second member at the intersection of the first member and second member. In some embodiments, the first member intersects and is coupled to the second member in a v-shape. In some embodiments, the first member non-intersectingly terminates proximate to the second member. In some embodiments, the wicking member is a fin-shaped member. In some embodiments, the wicking member includes an edge portion proximate to the wall of the reservoir bowl, wherein the edge portion comprises a reduced thickness.

One aspect of the present disclosure relates to a method of using a medication inhaler. The method includes inserting one or more drops of liquid into a reservoir bowl of a medication inhaler. The medication inhaler can include a mouth piece coupled to a bottom of a reservoir bowl, which reservoir bowl can include the bottom, a top, and a wall extending between the bottom and the top. The medication inhaler can include a wicking member extending at least partially into the reservoir bowl. The method can include wicking at least a portion of the drop of liquid with the wicking member to the bottom of the reservoir bowl, and generating inhalable droplets from the drop of liquid.

In some embodiments, the medication inhaler includes a mesh positioned at the bottom of the reservoir bowl. In some embodiments, generating inhalable droplets from the drop of liquid includes vibrating the mesh to generate the inhalable droplets from the portion of the drop of liquid at the bottom of the reservoir bowl. In some embodiments, the medication inhaler further includes a cap extending across the top of the reservoir bowl. In some embodiments, the cap defines an aperture through which the drop of liquid is inserted into the reservoir bowl. In some embodiments, at least the portion of the drop of liquid wicked to the bottom of the reservoir bowl can be wicked from a junction of the wall of the reservoir bowl and the cap.

In some embodiments, the wicking member connects to the wall of the reservoir bowl proximate to the top of the reservoir bowl. In some embodiments, the cap can have a rounded diamond shape with a major axis extending between a cap front and a cap back and a minor axis extending between a cap first side and a cap second side. In some embodiments, the wicking member is coupled to the bottom of the cap. In some embodiments, the wicking members extends along the major axis of the cap from the cap front to the cap back. In some embodiments, the wicking member at least partially obstructs the aperture defined by the cap. In some embodiments, the wicking member extends across the aperture defined by the cap without obstructing the aperture defined by the cap. In some embodiments, the medication inhaler includes a mouthpiece cartridge and a body, the mouthpiece cartridge including the reservoir bowl and the wicking member. In some embodiments, the method further includes inserting mouthpiece cartridge into the body.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

FIG. 1 is a perspective view of one embodiment of the medication inhaler.

FIG. 2 is a perspective view of one embodiment of filing a medication into the medication inhaler.

FIG. 3 is a section view of one embodiment of a mouthpiece cartridge without a wicking feature.

FIG. 4 is a top view of one embodiment of the mouthpiece cartridge.

FIG. 5 is a section view of one embodiment of the reservoir bowl containing drop of a medication without the wick.

FIG. 6 is a section view of one embodiment of a wicking member in the reservoir bowl.

FIG. 7 is a section view of another embodiment of the wicking member in the reservoir bowl.

FIG. 8 is a section view of a fin-shaped embodiment of the wicking member in the reservoir bowl.

FIG. 9 is a top view of one embodiment of the mouthpiece cartridge showing the position of a portion of the medication.

FIG. 10 is a section view of the reservoir bowl and wicking member showing wicking.

FIG. 11 depicts a series of side-section views and front-section views of one embodiment of the mouthpiece cartridge showing the movement of a portion of a drop moving toward a bottom of the reservoir bowl.

FIG. 12 is a graph depicting the performance effect of the wicking member.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides illustrative embodiment(s) only and is not intended to limit the scope, applicability, or configuration of the disclosure. Rather, the ensuing description of the illustrative embodiment(s) will provide those skilled in the art with an enabling description for implementing a preferred exemplary embodiment. It is understood that various changes can be made in the function and arrangement of elements without departing from the spirit and scope as set forth in the appended claims.

A medication inhaler can provide effective and easy delivery of medication to a patient. However, such devices, while easing the administration of a medication, can make the accurate administration of a dose of the medication subject to more variability. Specifically, in some instances only a portion of the medication may be administered to the patient. This can occur, for example, because the a portion of the medication is not converted into a mist, but rather remains within a reservoir of the medication inhaler.

Some embodiments of the present disclosure relate to a medication inhaler that can include a mouthpiece cartridge that can be received into a body. The mouthpiece cartridge can include a reservoir bowl that defines a reservoir in which medication can be contained. This medication can, in some embodiments, be added to the reservoir bowl immediately before administration of the medication. In such an embodiment, a dropper can access the reservoir bowl, in some embodiments, via an aperture in a cap located at a top of the reservoir bowl. In some embodiments, the dropper can be inserted through a portion of the body of the medication inhaler to allow access to the reservoir bowl.

When medication is added to the reservoir bowl, or when medication is contained in the reservoir bowl, some or all of the medication can accumulate at the bottom of the reservoir bowl. However, in some embodiments, all or portions of the medication can remain in parts of the reservoir bowl, other than the bottom. In some instances, for example, one or several droplets, which can comprise, some or all of the medication in the reservoir bowl, can adhere to a wall of the reservoir bowl. In some instances these droplets hang up and are not converted into inhalable mist through a vibrating mesh.

In some embodiments, the medication can include significant amounts of water, and in some embodiments, can be 96-99% water. Because of this high water percentage, the medication can fall into a class of liquids with properties dominated by bulk water. Thus, while the surface tension and viscosity of the medication may be modified by the drug and excipients, the medication can have properties primarily influenced by water. Due to this, the medication can have high surface tension that can result in adhesion of all or portions of the medication to walls of the reservoir bowl instead of moving to the bottom of the reservoir bowl. This adhesion can be affected by use of select materials such as ABS or polystyrene, however, even with such materials, adhesion forces, particularly at the junction of the wall of the reservoir bowl with the cap can be sufficiently high to hold a portion of the medication from the bottom of the reservoir bowl. Specifically, for example, the intersection of the cap and the reservoir wall can create a capillary space that can hold droplets ranging from 10-140 μl.

The present disclosure relates to a wicking member that extends into the reservoir bowl. This wicking member can be made of a variety of materials and be a variety of shapes and sizes. The wicking member can contact all or portions of one or several droplets adhering to the wall of the reservoir bowl, and can thereby change the shape of the droplet to reduce the contact area of the droplet, thereby lowering the adhesion forces to the wall. This decrease in the adhesion force can be sufficient such that gravity and liquid flow through the mesh in the bottom of the reservoir can pull the drop downward to the bottom of the reservoir bowl and the vibrating mesh.

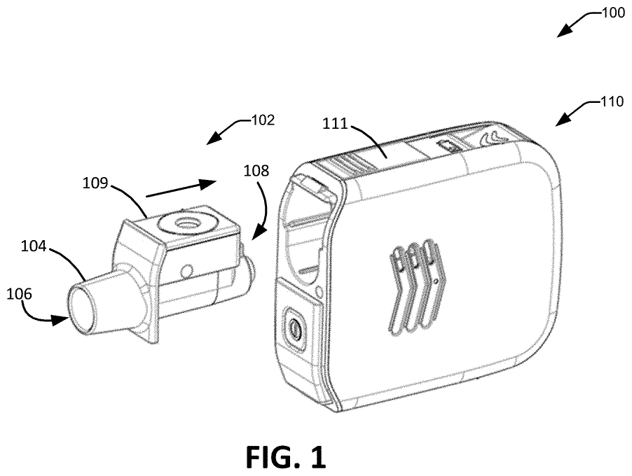

An embodiment of the medication inhaler 100 is shown in FIG. 1. The medication inhaler 100, as discussed above, can include the mouthpiece cartridge 102. The mouthpiece cartridge can include a mouthpiece 104 that can be located at a front 106 of the mouthpiece cartridge 100 and a back 108 located opposite of the front 106 and/or of the mouthpiece 104. The mouthpiece further includes a reservoir assembly 109 that can be, in some embodiments, located intermediate between the mouthpiece 104 and the back 108 and/or intermediate between the front 106 and the back 108 of the mouthpiece cartridge 102. The mouthpiece cartridge 102 can be coupled to and/or received in a body 110. In some embodiments, the mouthpiece cartridge 102 can be inserted into the body 110. The body 110 can include a slider 111 that can be retracted to allow access to the reservoir assembly 109 when the mouthpiece cartridge 102 is received in the body 110.

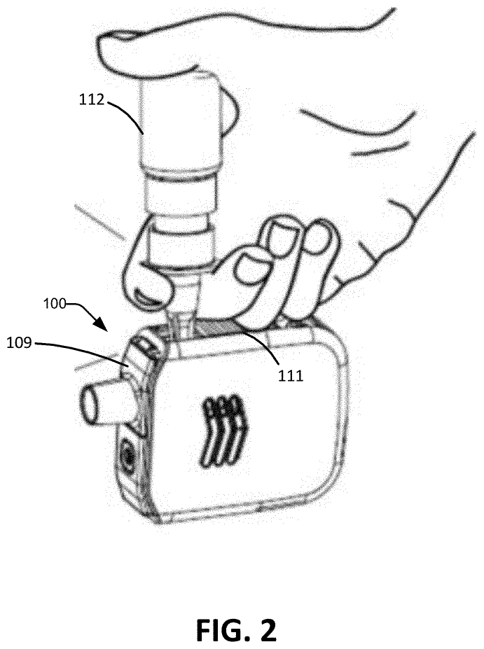

As seen in FIG. 2, medication can be provided to the medication inhaler 100. The medication inhaler 100 can be filled with medication, and/or can contain medication. In some embodiments, the medication inhaler 100 can be filled with medication via a dropper 112. In some embodiments, the dropper 112 can meter drops to thereby control dosing. The dropper 112 can be inserted into and/or can be positioned proximate to the medication inhaler 100, and specifically proximate to the mouthpiece cartridge 102. In some embodiments, it is possible to load the mouthpiece cartridge 102 while the mouthpiece cartridge is detached from the body 110. In some embodiments, the dropper 112 can be used to provide one or several droplets of medication to the medical inhaler. In some embodiments, and as part of filling medication, the slider 111 can be retracted and then the dropper 112 can be inserted into and/or can access the reservoir assembly 109.

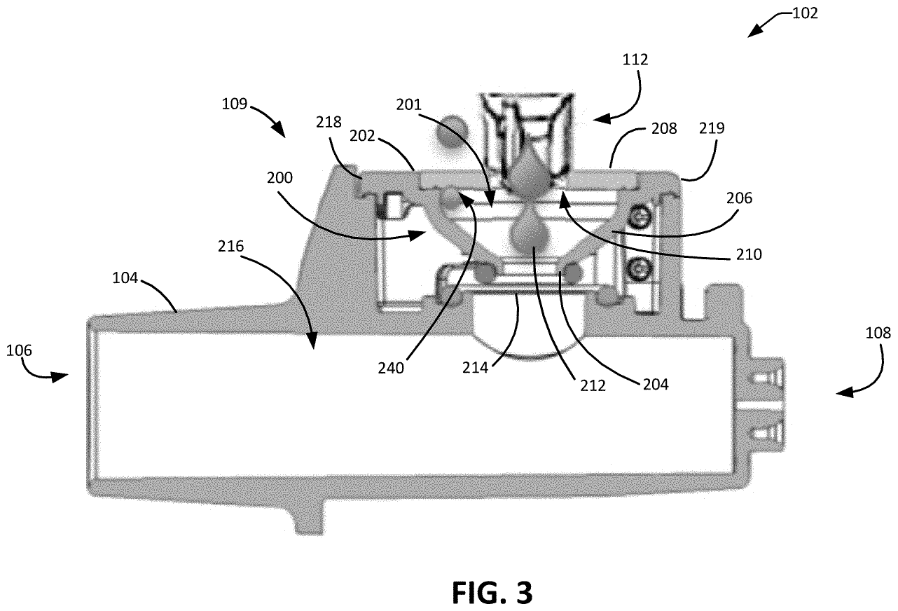

As seen in FIG. 3, the reservoir assembly 109 can include a reservoir bowl 200 that includes a top 202, a bottom 204, and a wall 206 extending between the top 202 and the bottom 204. The reservoir bowl 200 defines a reservoir 201 in which a volume of medication can be received. The reservoir bowl 200 has a front 218 and a back 219. The front 218 is positioned relatively proximate to the front 106 of the mouthpiece cartridge 102. The back 219 is positioned relatively proximate to the back 108 of the mouthpiece cartridge 102.

A cap 208 is positioned at the top 202 of the reservoir bowl 200, and specifically is received within the top 202 of the reservoir bowl 200. The intersecting of the cap 208 and the reservoir bowl 200 can create a junction 230. In some embodiments, and as seen in FIG. 3, the cap 208 can extend across the top 202 of the reservoir bowl 200. The cap 208 defines an aperture 210 through which medication can be filled into the reservoir 201. As seen in FIG. 3, the dropper 112 can fill medication into the reservoir 201 via the aperture 210, and specifically the dropper 112 can fill medication into the reservoir 201 in the form of one or more drops 212.

The reservoir assembly 109 can include a mesh 214 that can be located at the bottom 204 of the reservoir bowl 200. The mesh 214 can be vibrated to thereby generate an inhalable mist. Specifically, medication located at the bottom 204 of the reservoir bowl 200 can be contact and/or be in contact with the mesh 214. Upon vibration, the mesh 214 can generate mist, which can be made of a plurality of inhalable droplets, which can be delivered to a lumen 216 connecting reservoir bowl 200 and the mesh 214 to the mouthpiece 104 of the mouthpiece cartridge 102. A patient can use the mouthpiece 104 to inhale the mist, thereby receiving the medication and/or a dose of the medication.

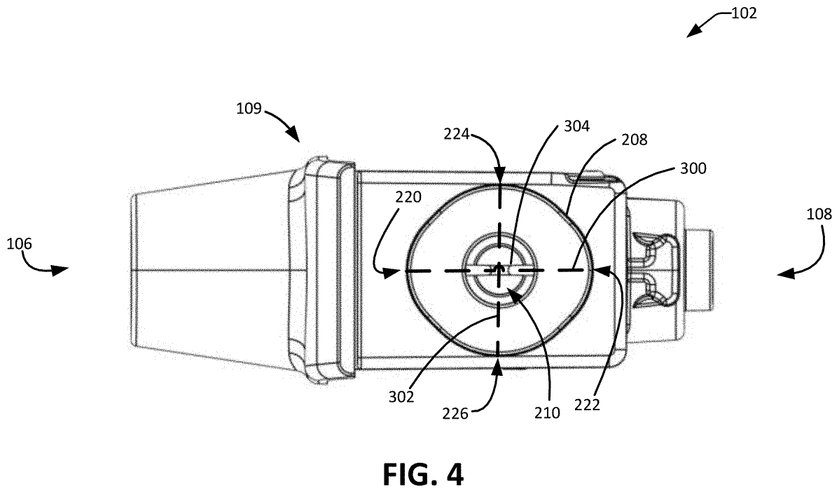

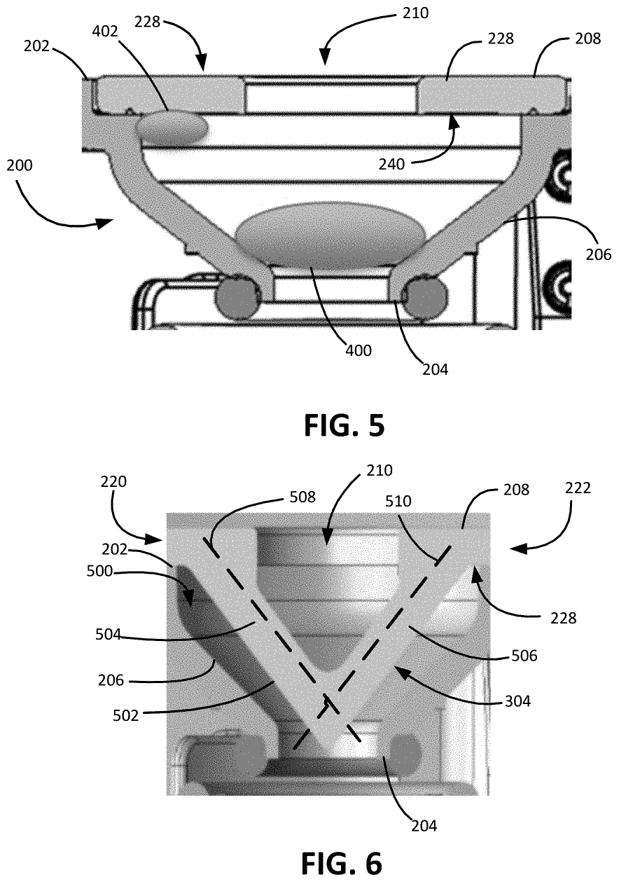

As seen in FIG. 4, the cap 208 has a cap front 220, a cap back 222, a cap first side 224, and a cap second side 226. As seen in FIG. 5, the cap 208 further includes a cap top 228 and a cap bottom 240. The cap 208 can be any desired size and shape, including circular, oblong, diamond, rectangular, or the like. In the embodiment shown in FIG. 4, the cap 208 has a rounded diamond shape. This rounded diamond-shaped cap 208 has a major axis 300 and a minor axis 302. The major axis 300 extends from the cap front 220 to the cap back 222 and the minor axis extends from the cap first side 224 to the cap second side 226. As seen in FIG. 4, the major axis 300 is greater, or more specifically, longer than the minor axis 302. As further seen, the cap 208 is received in the top of the reservoir bowl 200 such that the major axis 300 of the cap extends from the front 218 of the reservoir bowl 200 to the back 219 of the reservoir bowl 200.

When medication, and specifically when a drop 212 of medication is dispensed into the reservoir bowl 200, the medication, and specifically the drop 212 of medication usually lands on the bottom 204 of the reservoir bowl 200. In some instances, because of splashing and other forces, and as seen in FIG. 5, some or all of the medication may not rest on the bottom 204 of the reservoir bowl 200. Specifically, FIG. 5 shows a first portion 400 of the drop 212 as having fallen to the bottom 204 of the reservoir bowl 200, and a second portion 402 of the drop 212 adhering to the wall 206 of the reservoir bowl 200, and specifically adhering to both the wall of the reservoir bowl 200 and the cap 208 at the junction 240 of the cap 208 and the reservoir bowl 200 wall 206. Although depicted as a single second portion 402, in some embodiments, the second portion can comprise one or several independent portions located at one or several different locations of the cap 208. In some instances, the adhesion forces of the second portion 402 of the drop 212 overcome gravitational forces and prevent the second portion 402 of the drop 212 from falling to the bottom 204 of the reservoir bowl 200.

As seen in FIG. 4, in some embodiments, the medication inhaler 100 can include a wicking member 304. The wicking member 304 can be configured to interact with the drop 212, and specifically with portions of the drop 212 that have not fallen to the bottom 204 of the reservoir bowl 200. Specifically, the wicking member 304 can be configured to interact with the second portion 402 of the drop 212. As a result of this interaction, the adhesion forces acting on the second portion 402 can be minimized, allowing the second portion 402 of the drop 212 to flow to the bottom 204 of the reservoir bowl 200. As seen in FIG. 4, the wicking member 304 extends along the major axis 300 of the cap 208 from the cap front 220 to the cap back 222.

The wicking member 304 can comprise a variety of shapes and sizes and can be made from a variety of materials. In some embodiments, the wicking member can be made from a material with low adhesion to the medication. This material can include, for example, “acrylonitrile butadiene styrene, polycarbonate, or polystyrene. In some embodiments, the wicking member 304 can be sized to minimize impact on the volume of the reservoir 201. Specifically, the wicking member 304, in combination with the reservoir bowl 202, can be sized to allow the reservoir 201 to hold all volumes within a desired range of volumes, and specifically to hold a largest desired dosage.

As seen in FIG. 6, the wicking member 304 can extend into the reservoir bowl 200, and in some embodiments, can extend into the reservoir bowl 200 towards the bottom 204 of the reservoir bowl 200. In some embodiments, the wicking member 304 can extend at least partially from the top 202, or proximate to the top 202 of the reservoir bowl 200 to the bottom 204 of the reservoir bowl 200.

The wicking member 304 can be positioned proximate to the wall 206 of the reservoir bowl. As seen in FIG. 6, this positioning of the wicking member 304 proximate to the wall 206 of the reservoir bowl 200 leaves a gap 500 between the wicking member 304 and the reservoir bowl 200. The wicking member 304 can include an edge portion 502. The edge portion 502 can be proximate to the wall 206 of the reservoir bowl 200. The edge portion 502 can have a reduced thickness. The gap 500 can be between the edge portion 502 of the wicking member 304 and the wall 206 of the reservoir bowl 200.

In some embodiments, the wicking member 304 can connect to the wall 206 of the reservoir bowl 200 proximate to the top 202 of the reservoir bowl 200. In some embodiments, the wicking member 304 can be coupled to the bottom 228 of the cap 208. The wicking member 304 can extend across the aperture 210. In some embodiments, the wicking member 304 at least partially obstructs the aperture 210 defined by the cap 208. The wicking member 304 at least partially obstructs the aperture 210 when a portion of the wicking member 304 inside of the area of the aperture 210 is at or above the level of the bottom 204 of the cap 208. Alternatively, and as depicted in FIG. 6, in some embodiments, the wicking member extends across the aperture 210 without partially obstructing the aperture 210. As seen in FIG. 3, embodiments in which the aperture 210 is not obstructed can advantageously allow a portion of the dropper 112, such as, for example, a nozzle, to enter into the reservoir 201 via the aperture 210, thereby facilitating the placement of medication inside of the reservoir 201.

As seen in FIG. 6, the connection of the wicking member 304 and the cap and/or the wall 206 can include one or several fillets or rounds. These fillets or rounds can increase the strength of the wicking member 304, but can also eliminate sharp edges, which can result in increased performance in wicking medication to the bottom 204 of the reservoir bowl 202.

The wicking member 304 can comprise a single member, or the wicking member 304 can comprise a plurality of members. As depicted in FIG. 6, the wicking member 304 comprises a first member 504 and a second member 506. In FIG. 6, the first member 504 extends from the cap 208, and specifically from, or proximate to the cap front 220 and the second member 506 extends from the cap 208, and specifically from, or proximate to the cap back 222.

The first member 504 can extend in a first direction as indicated by axis 508 and the second member 506 can extend in a second direction as indicated by axis 510. In some embodiments, the edge portion 502 of the first member 504 extends in the first direction and the edge portion 502 of the second member 506 extends in the second direction. In some embodiments, the first direction can be parallel with the second direction, can be away from the second direction, and in some embodiments, the first direction can extend at least partially towards the second direction. Thus, in some embodiments, the first member 504, or the edge portion 502 of the first member 504 can be parallel, extending away from, or at least partially extending towards the second member 506, or the edge portion 502 of the second member 506.

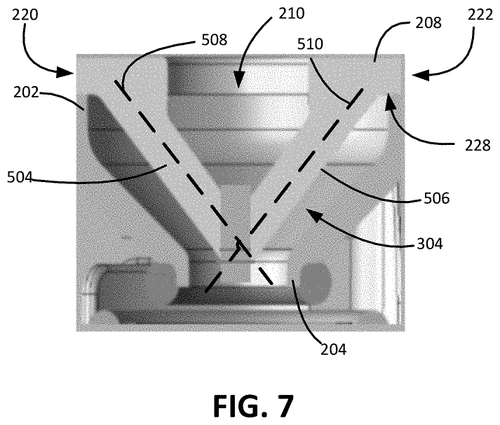

In some embodiments, and as shown in FIG. 7 the first member 504 and the separate member 506 can be non-intersecting, and non-coupling, except for through the cap 208. In some such embodiments, the first member 504 can extend towards the second member 506, but can terminate before intersection. In such an embodiment, the first member 504 can terminate proximate to the second member 506.

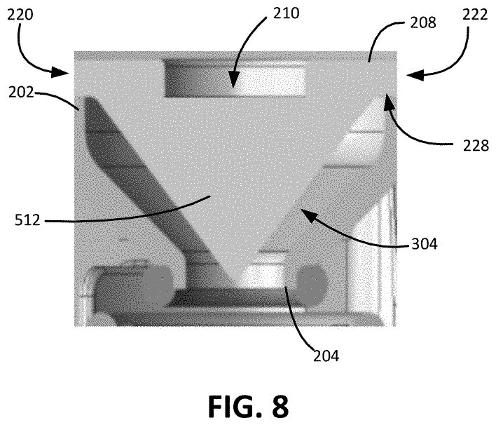

In other embodiments, and as depicted in FIG. 6, in some embodiments, the first member 504 intersects with the second member 506, and the first member 504 is coupled to the second member 506 at the intersection of the first and second members 504, 506. In some embodiments, the wicking member 304 can be v-shaped as shown in FIG. 6, or more specifically, the intersection and coupling of the first and second members 504, 506 can create a v-shaped wicking member 304. Alternatively, and as depicted in FIG. 8, in some embodiments, the wicking member 304 can comprise a single member and can comprise a fin-shaped member 512.

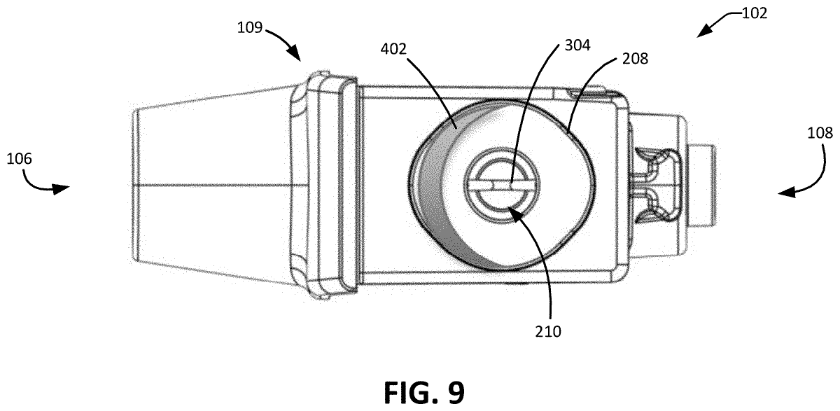

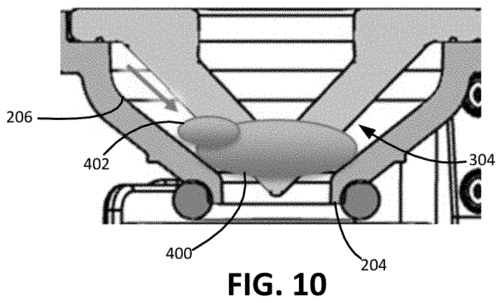

In some embodiments, the wicking member can contact the second portion 402 of the drop 212 to thereby wick the second portion 402 of the drop 212 to the bottom 204 of the reservoir bowl 202. In some instances, the second portion 402 of the drop 212 can extend around a portion of the reservoir bowl 202 in a crescent shape, as indicated in FIG. 9. As shown in FIG. 10, the wicking member 304 can contact all or a portion of the second portion 402 of the drop 212. Through this contact, the wicking member 304 can deform the second portion 402 of the drop 212, decreasing the contact of the second portion 402 of the drop 212 with all or portions of the wall 206 and the cap 208 and allowing gravity to pull the second portion 402 of the drop 212 to the bottom 204 of the reservoir bowl 202. The combination of the small size of the wicking member 304, compared to the wall 206 and the downward extension of the wicking member 304 allows gravity to pull the medication on the wicking member 304 to the bottom 204 of the reservoir bowl 202. Thus, the wicking member 304 can wick all or portions of the second portion 402 of the drop 212 to the bottom 204 of the reservoir bowl 202. In some embodiments, the second portion can comprise one or more independent portions which can located at different portions of the cap 208. In embodiment in which the second portion 402 of the drop 212 contacts both the wall 206 and the cap 208, the wicking member 304 can wick the second portion 402 of the drop 212 from the junction 240 of the wall 206 of the reservoir bowl 202 and the cap 208. In embodiments in which the second portion 402 comprises a plurality of independent portions, some or all of the independent portions can be independently wicked to the bottom 204 of the reservoir bowl 202.

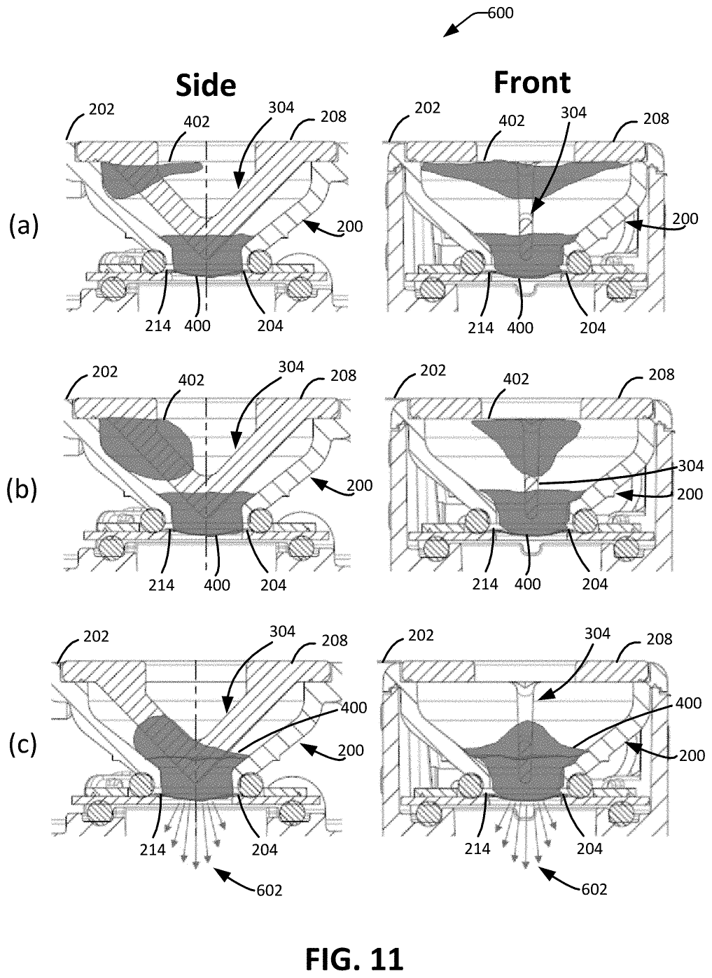

The wicking of the second portion 402 of the drop 212 to the bottom 204 of the reservoir bowl 200 can be seen in FIG. 11. FIG. 11 depicts a series of views 500, and specifically a series of side-section and front-section views of one embodiment of the reservoir bowl 200. This series of views 600 shows the movement of a portion, and specifically of the second portion 402 of the drop 212 moving toward a bottom 204 of the reservoir bowl 200. As seen in the front and side views in row (a), the second portion 402 of the drop 212 is located at the top 202 of the reservoir bowl 200, and the second portion 402 of the drop 212 is beginning to wick towards the bottom 204 of the reservoir bowl 200. This wicking is further seen in the front and side views in row (b). As seen in the front and side views in row (c), the second portion 402 of the drop 212 has merged with the first portion 400 of the drop 212. As further seen in the views in row (c), the drop 212 is in contact with the mesh 214, which mesh 214 can be vibrated to thereby generate inhalable mist 602. Although the generation of the inhalable mist 602 is only shown in the view of row (c), this inhalable mist can be generated at any time when a portion of drop 212 is in contact with the mesh 214.

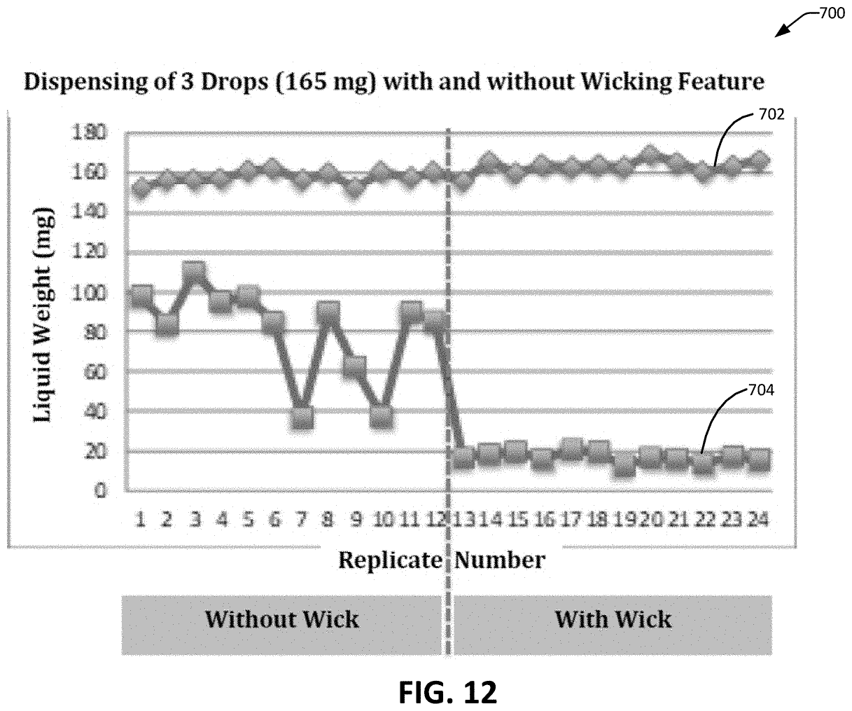

FIG. 12 is a graph 700 depicting amount of medication placed in a reservoir bowl 202 with line 702 and medication remaining in the mouthpiece cartridge 102 after medication delivery with line 704. The medication remaining in the mouthpiece cartridge 102 includes both medication remaining in the reservoir bowl 202 and in the lumen 216 of the mouthpiece cartridge 102. As seen in FIG. 12, the inclusion of the wicking member 304 in the medication inhaler 100 significantly improves performance of the medication inhaler 100. More specifically, without the wicking member 304, the medication inhaler consistently fails to delivery approximately 50% of the medication in the reservoir bowl 202. In contrast to this, the wicking member 304 decreased the amount of the undelivered medication from approximately 50% to approximately 12%.

In the foregoing specification, the invention is described with reference to specific embodiments thereof, but those skilled in the art will recognize that the invention is not limited thereto. Various features and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

What is claimed is:

1. A medication inhaler comprising:
a reservoir bowl defining a reservoir wherein a volume of liquid can be received, the reservoir bowl comprising a top, a bottom, and a wall extending from the top to the bottom of the reservoir bowl; and
a wicking member proximate the wall, and the wicking member extending along the wall at least partially from the top of the reservoir bowl to the bottom of the reservoir bowl, wherein the wicking member is configured to wick liquid to the bottom of the reservoir bowl.

2. The medication inhaler of claim 1, wherein the wicking member connects to the wall of the reservoir bowl proximate to the top of the reservoir bowl.

3. The medication inhaler of claim 1, further comprising a cap defining an aperture, the cap extending across the top of the reservoir bowl.

4. The medication inhaler of claim 3, further comprising a mouthpiece at a front of the medication inhaler and an opposing back of the medication inhaler.

5. The medication inhaler of claim 4, wherein the cap comprises a rounded diamond shape with a major axis extending between a cap front and a cap back and a minor axis extending between a cap first side and a cap second side, wherein the major axis is greater than the minor axis, and wherein the cap is received in the top of the reservoir bowl.

6. The medication inhaler of claim 5, wherein the reservoir bowl comprises a front proximate to the mouthpiece and a back proximate to the back of the medication inhaler, and wherein the cap is received in the top of the reservoir bowl such that the major axis of the cap extends from the front of the reservoir bowl to the back of the reservoir bowl.

7. The medication inhaler of claim 6, wherein the wicking member is coupled to the bottom of the cap.

8. The medication inhaler of claim 7, wherein the wicking member extends along the major axis of the cap from the cap front to the cap back.

9. The medication inhaler of claim 1, wherein the wicking member comprises a single member.

10. The medication inhaler of claim 1, wherein the wicking member comprises a plurality of members.

11. The medication inhaler of claim 1, wherein the wicking member comprises a first member extending from a cap front and a second member extending from a cap back, wherein the first member extends at least partially towards the second member.

12. The medication inhaler of claim 11, wherein the first member intersects the second member, and wherein the first member is coupled to the second member at the intersection of the first member and second member.

13. The medication inhaler of claim 12, wherein the first member intersects and is coupled to the second member in a v-shape.

14. The medication inhaler of claim 12, wherein the first member non-intersectingly terminates proximate to the second member.

15. The medication inhaler of claim 12, wherein the wicking member comprises a fin-shaped member.

16. The medication inhaler of claim 15, wherein the wicking member comprises an edge portion proximate to the wall of the reservoir bowl, wherein the edge portion comprises a reduced thickness.

17. A method of using a medication inhaler, the method comprising:
inserting a drop of liquid into a reservoir bowl of a medication inhaler, the medication inhaler comprising a mouth piece coupled to a bottom of a reservoir bowl, the reservoir bowl comprising the bottom, a top, and a wall extending between the bottom and the top, and a wicking member extending at least partially into the reservoir bowl;
wicking at least a portion of the drop of liquid with the wicking member to the bottom of the reservoir bowl; and
generating inhalable droplets from the drop of liquid.

18. The method of claim 17, wherein the medication inhaler comprises a mesh positioned at the bottom of the reservoir bowl.

19. The method of claim 18, wherein generating inhalable droplets from the drop of liquid comprises vibrating the mesh to generate the inhalable droplets from the portion of the drop of liquid at the bottom of the reservoir bowl.

20. The method of claim 19, wherein the medication inhaler further comprises a cap extending across the top of the reservoir bowl, wherein the cap defines an aperture where through the drop of liquid is inserted into the reservoir bowl.

21. The method of claim 20, wherein the at least the portion of the drop of liquid wicked to the bottom of the reservoir bowl are wicked from a junction of the wall of the reservoir bowl and the cap.

22. The method of claim 21, wherein the wicking member connects to the wall of the reservoir bowl proximate to the top of the reservoir bowl.

23. The method of claim 21, wherein the cap comprises a rounded diamond shape with a major axis extending between a cap front and a cap back and a minor axis extending between a cap first side and a cap second side.

24. The method of claim 23, wherein the wicking member is coupled to the bottom of the cap.

25. The method of claim 24, wherein the wicking members extends along the major axis of the cap from the cap front to the cap back.

26. The method of claim 25, wherein the wicking member at least partially obstructs the aperture defined by the cap.

27. The method of claim 25, wherein the wicking member extends across the aperture defined by the cap without obstructing the aperture defined by the cap.

28. The method of claim 17, the medication inhaler comprising a mouthpiece cartridge and a body, the mouthpiece cartridge including the reservoir bowl and the wicking member, the method further comprising inserting mouthpiece cartridge into the body.
